(19) United States
(12) Patent Application Publication
(10) Pub. No.: US 2020/0101093 A1
(43) Pub. Date: Apr. 2, 2020

(54) Methods of Treating and Preventing Neuropsychiatric and/or Neurocognitive Complications in Patients Infected with HIV

(71) Applicant: Janssen Sciences Ireland Unlimited Company, Co Cork (IE)

(72) Inventors: Aikaterini Pavlaki, Athens (GR); Asimina Zavitsanou, Athens (GR)

(21) Appl. No.: 16/586,371

(22) Filed: Sep. 27, 2019

Related U.S. Application Data
(60) Provisional application No. 62/751,354, filed on Oct. 26, 2018.
(60) Provisional application No. 62/738,316, filed on Sep. 28, 2018.

(51) Int. Cl. A61K 31/683; A61K 31/34; A61P 25/00

(52) U.S. Cl. CPC A61K 31/683; A61P 25/00; A61K 31/34

(57) ABSTRACT

The disclosure is directed to methods of treating humans diagnosed with an HIV infection and who are also diagnosed with a neuropsychiatric and/or neurocognitive complication of an HIV infection or who are also predisposed to a neuropsychiatric and/or a neurocognitive complication of an HIV infection.

### TECHNICAL FIELD

The disclosure is directed to methods of treating humans diagnosed with an HIV infection and who are also diagnosed with neuropsychiatric and/or neurocognitive complications of an HIV infection or who are also predisposed to neuropsychiatric and/or neurocognitive complications of an HIV infection.

### BACKGROUND

Human immunodeficiency virus (HIV) infection is often preceded or accompanied by neuropsychiatric conditions, including mood and personality disorders and psychosis. Mental illness, in conjunction with HIV in patients, worsens functional impairment and quality of life, and is associated with rapid and harder to treat progression of HIV disease. Depression is the most common neuropsychiatric complication of HIV, with a 2- to 4-fold increase in the prevalence of major depressive disorder compared with that of the general population. Anxiety disorders are also notably higher among HIV infected patients than the general population. Moreover, sleep disturbances and fatigue are common in HIV populations.

HIV-associated neurocognitive disorder (HAND) is the range of neurocognitive dysfunction associated with HIV infection, and includes asymptomatic neurocognitive impairment, mild neurocognitive disorder and HIV-associated dementia (HAD). The prevalence of HAD, which is invariably fatal, has declined substantially over time, but milder forms of HAND remain common within HIV-infected adults.

In HIV patients, recognition of the clinical manifestations of mental disorders is a major challenge. Additionally, late recognition of neuropsychiatric disorders is related, among other things, with diminished coping capacity at diagnosis, failure at primary prevention, poor antiviral adherence, impairment in quality of life, greater social burden, overall increases in healthcare costs, and increased morbidity and mortality. As a result, regular health screening is already endorsed by current European AIDS Clinical Society (EACS) guidelines. But despite being frequent, these disorders are often underdiagnosed and undertreated.

Many factors may be implicated in the development of mental disorders in HIV patients. These include, but are not limited to, demographic, genetic and social factors, aging, current occupation, marital status, smoking, alcohol use, neurobiological changes related to the persistent viral presence in the central nervous system, reaction to social stigma and sexual dysfunction, coping with the prospect of illness and death, side effects of antiretroviral therapy (ART), HCV co-infection, comorbidities and associated treatments.

Additionally, research has indicated that stressful life events are prevalent in HIV-infected patients. While the majority are unrelated to HIV disease, these events can have a great impact on the course of HIV disease progression, exacerbating physical and psychological symptoms, and negatively affecting treatment outcomes.

The numerous neuropsychiatric complications of HIV make it difficult to identify which are related to the antiretroviral therapy (ART), and which are related to the virus itself. Where ART is concerned, the frequency and severity of neuropsychiatric adverse effects is highly variable, with differences between the antiretroviral classes and amongst the individual drugs in each class. Neuropsychiatric adverse effects are most commonly seen with nucleoside reverse transcriptase inhibitors, nucleotide reverse transcriptase inhibitors (NRTIs), and non-nucleoside reverse-transcriptase inhibitors (NNRTIs), and even with some of the antiretrovirals prescribed worldwide such as efavirenz. Similar results are confirmed by others, underscoring the need for further investigation, especially in populations usually underrepresented in clinical trials.

Methods of treating or preventing one or more neuropsychiatric and/or neurocognitive complications of HIV infection are required.

### SUMMARY

The disclosure is directed to methods of treating humans diagnosed with an HIV infection, who are also diagnosed with neuropsychiatric and/or neurocognitive complications of an HIV infection or who are also predisposed to a neuropsychiatric and/or neurocognitive complication of an HIV infection. The methods comprise administering darunavir, a darunavir solvate, or a darunavir hydrate; cobicistat; tenofovir a tenofovir prodrug, a pharmaceutically acceptable salt of tenofovir, or a pharmaceutically acceptable salt of a tenofovir prodrug; and emtricitabine.

### DETAILED DESCRIPTION OF ILLUSTRATIVE EMBODIMENTS

The present disclosure may be understood more readily by reference to the following detailed description taken in connection with the examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. As used herein.

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically carriers, and excludes other compounds.

All ranges disclosed herein are inclusive of the recited endpoint and independently combinable (for example, the range of “from 2 mg to 10 mg” is inclusive of the endpoints, 2 mg and 10 mg, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9 to 1.1. Other meanings of “about” may be apparent from the context, such as rounding off, so, for example “about 1” may also mean from 0.5 to 1.4.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. See, e.g., U.S. Food & Drug Administration, Pharmaceutical Quality/CMC Guidances.

“Pharmaceutically acceptable excipient” refers to a diluent, adjuvant, excipient or carrier with which a compound of the disclosure is administered. A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, stearates, silicon dioxide, polyvinyl alcohols, talc, titanium dioxide, ferric oxide, and polyethylene glycols. See, e.g., U.S. Food & Drug Administration, Pharmaceutical Quality/CMC Guidances.

“Subject” includes humans. The terms “human,” “patient,” and “subject” can be used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Single unit dosage form” as used herein refers to dosage forms suitable for oral administration such as, but not limited to, tablets, capsules, gelcaps, and caplets.

The disclosure is directed to methods of treating a human diagnosed with an HIV infection and who is also diagnosed with a neuropsychiatric complication of an HIV infection. The disclosure is also directed to methods of treating a human diagnosed with an HIV infection and who, while not diagnosed with a neuropsychiatric complication, is identified as being predisposed to a neuropsychiatric complication of an HIV infection.

The disclosure is also directed to methods of treating a human diagnosed with an HIV infection and who is also diagnosed with a neurocognitive complication of an HIV infection. The disclosure is also directed to methods of treating a human diagnosed with an HIV infection and who, while not diagnosed with a neurocognitive complication, is identified as being predisposed to a neuropsychiatric complication of an HIV infection.

The disclosure is directed to methods of treating a human diagnosed with an HIV infection and who is also diagnosed with a neuropsychiatric complication of an HIV infection and is also diagnosed with a neurocognitive complication of an HIV infection. The disclosure is also directed to methods of treating a human diagnosed with an HIV infection and who, while not diagnosed with a neuropsychiatric complication or a neurocognitive complication of an HIV infection, is identified as being predisposed to a neuropsychiatric complication and identified as being predisposed to a neurocognitive complication of an HIV infection.

Methods of diagnosing an HIV infection are known in the art and include ELISA tests, home tests, saliva tests, viral load tests, and Western Blots.

In some aspects, the disclosure is directed to methods of treating a human diagnosed with an HIV infection, for example, an HIV-1 infection, and who is also diagnosed with a neuropsychiatric complication of an HIV infection. Neuropsychiatric complications of an HIV infection include, for example, depressive spectrum disorders including major depressive disorder, mania, psychosis, anxiety including generalized anxiety disorder, social phobia, fatigue including chronic fatigue, sleep disturbance including disorders of initiating and/or maintaining sleep, disorders of excessive somnolence, disorders of sleep-wake schedule, and dysfunctions associated with sleep, sleep stages, or partial arousals; and poor sleep quality. In some aspects, the neuropsychiatric complication of an HIV infection is a diagnosis of one or more of depressive spectrum disorders including major depressive disorder, mania, psychosis, anxiety including generalized anxiety disorder, social phobia, fatigue including chronic fatigue, sleep disturbance including disorders of initiating and/or maintaining sleep, disorders of excessive somnolence, disorders of sleep-wake schedule, and dysfunctions associated with sleep, sleep stages, or partial arousals; and poor sleep quality. In other aspects, the neuropsychiatric complication of an HIV infection is a diagnosis of one or more of depression, anxiety, fatigue, and sleep disturbances. In some aspects, the human is diagnosed with one neuropsychiatric complication of an HIV infection. In other aspects, the human is diagnosed with two neuropsychiatric complications of an HIV infection. In yet other aspects, the human is diagnosed with three or more neuropsychiatric complications of an HIV infection.

For example, the human may be diagnosed with an HIV infection, for example an HIV-1 infection, and a depressive spectrum disorder such as major depressive disorder. In other aspects, the human may be diagnosed with an HIV infection, for example an HIV-1 infection, and mania. In other aspects, the human may be diagnosed with an HIV infection, for example an HIV-1 infection, and psychosis. In other aspects, the human may be diagnosed with an HIV infection, for example an HIV-1 infection, and an anxiety disorder, for example, generalized anxiety disorder. In other aspects, the human may be diagnosed with an HIV infection, for example an HIV-1 infection, and social phobia. In other aspects, the human may be diagnosed with an HIV infection, for example an HIV-1 infection, and fatigue, such as chronic fatigue. In other aspects, the human may be diagnosed with an HIV infection, for example an HIV-1 infection, and sleep disturbance, such as, for example, a disorder of initiating and/or maintaining sleep, a disorder of excessive somnolence, a disorder of sleep-wake schedule, or a dysfunction associated with sleep, sleep stages, or partial arousal. In other aspects, the human may be diagnosed with an HIV infection, for example an HIV-1 infection, and poor sleep quality.

In some aspects, the disclosure is directed to methods of treating a human diagnosed with an HIV infection, for example, an HIV-1 infection, and who is also diagnosed with a neuropsychiatric complication of an HIV infection as described herein, and who is also diagnosed with a neurocognitive complication of an HIV infection. Neurocognitive complications of an HIV infection include, for example, asymptomatic neurocognitive impairment (ANI), mild neurocognitive disorder (MND) and HIV associated dementia (HAD).

In other aspects, the disclosure is directed to methods of treating a human diagnosed with an HIV infection, for example, an HIV-1 infection, and who is also predisposed to a neuropsychiatric complication of an HIV infection. Neuropsychiatric complications of an HIV infection include, for example, depressive spectrum disorders including major depressive disorder, mania, psychosis, anxiety including generalized anxiety disorder, social phobia, fatigue including chronic fatigue, sleep disturbance including disorders of initiating and/or maintaining sleep, disorders of excessive somnolence, disorders of sleep-wake schedule, and dysfunctions associated with sleep, sleep stages, or partial arousals; and poor sleep quality. In some aspects, the human is predisposed to one or more neuropsychiatric complications of an HIV infection, such as, for example, one or more of depressive spectrum disorders including major depressive disorder, mania, psychosis, anxiety including generalized anxiety disorder, social phobia, fatigue including chronic fatigue, sleep disturbance including disorders of initiating and/or maintaining sleep, disorders of excessive somnolence, disorders of sleep-wake schedule, and dysfunctions associated with sleep, sleep stages, or partial arousals; and poor sleep quality. In some aspects, the human is predisposed to one or more of depression, anxiety, fatigue, and sleep disturbances. In other aspects, the human is predisposed to two neuropsychiatric complications of an HIV infection. In yet other aspects, the human is predisposed to three or more neuropsychiatric complications of an HIV infection.

For example, the human may be diagnosed with an HIV infection, for example an HIV-1 infection, and be predisposed to a depressive spectrum disorder such as major depressive disorder. In other aspects, the human may be diagnosed with an HIV infection, for example an HIV-1 infection, and be predisposed to mania. In other aspects, the human may be diagnosed with an HIV infection, for example an HIV-1 infection, and be predisposed to psychosis. In other aspects, the human may be diagnosed with an HIV infection, for example an HIV-1 infection, and be predisposed to an anxiety disorder, for example, generalized anxiety disorder. In other aspects, the human may be diagnosed with an HIV infection, for example an HIV-1 infection, and be predisposed to social phobia. In other aspects, the human may be diagnosed with an HIV infection, for example an HIV-1 infection, and be predisposed to fatigue, such as chronic fatigue. In other aspects, the human may be diagnosed with an HIV infection, for example an HIV-1 infection, and be predisposed to sleep disturbance, such as, for example, a disorder of initiating and/or maintaining sleep, a disorder of excessive somnolence, a disorder of sleep-wake schedule, or a dysfunction associated with sleep, sleep stages, or partial arousal. In other aspects, the human may be diagnosed with an HIV infection, for example an HIV-1 infection, and be predisposed to poor sleep quality.

In some aspects, the disclosure is directed to methods of treating a human diagnosed with an HIV infection, for example, an HIV-1 infection, and who is also predisposed to a neuropsychiatric complication of an HIV infection as described herein, and who is also predisposed to a neurocognitive complication of an HIV infection. Neurocognitive complications of an HIV infection include, for example, asymptomatic neurocognitive impairment, mild neurocognitive disorder, and HIV-associated dementia (HAD).

Those of skill in the art are able to identify whether a human is predisposed to one or more neuropsychiatric and/or neurocognitive complications of an HIV infection using the knowledge and skills of the art. For example, some humans who have been diagnosed with an HIV infection, for example, an HIV-1 infection, may be genetically predisposed to one or more neuropsychiatric complications of an HIV infection. Some humans who have been diagnosed with an HIV infection, for example, an HIV-1 infection, may be genetically predisposed to one or more neurocognitive complications (for example, executive function, attention and concentration) of an HIV infection. Some humans who have been diagnosed with an HIV infection, for example, an HIV-1 infection, may have had a previous diagnosis of one or more of, for example, depressive spectrum disorder including major depressive disorder, mania, psychosis, anxiety including generalized anxiety disorder, social phobia, fatigue including chronic fatigue, a sleep disturbance including a disorder of initiating and/or maintaining sleep, a disorder of excessive somnolence, a disorder of sleep-wake schedule; and poor sleep quality. Such humans who have had such a previous diagnosis may be considered more susceptible to developing one or more neuropsychiatric complications of an HIV infection. Other humans diagnosed with an HIV infection, for example, an HIV-1 infection, may lack adequate social and/or emotional support, making them more susceptible to developing one or more neuropsychiatric complications of an HIV infection. Other humans diagnosed with an HIV infection, for example, an HIV-1 infection, may lack adequate financial resources, making them more susceptible to developing one or more neuropsychiatric complications of an HIV infection.

According to the disclosure, the HIV-infected human, whether diagnosed with a neuropsychiatric and/or neurocognitive complication of HIV infection or predisposed to a neuropsychiatric and/or neurocognitive complication of an HIV infection, is administered:
darunavir, a darunavir solvate, or a darunavir hydrate;
cobicistat;
tenofovir, a tenofovir prodrug, a pharmaceutically acceptable salt of tenofovir, or a pharmaceutically acceptable salt of a tenofovir prodrug; and
emtricitabine.

In some aspects, the HIV-infected human, whether diagnosed with a neuropsychiatric and/or neurocognitive complication of HIV infection or predisposed to a neuropsychiatric and/or neurocognitive complication of an HIV infection, is administered:
darunavir, a darunavir solvate, or a darunavir hydrate;
cobicistat;
a tenofovir prodrug or a pharmaceutically acceptable salt of a tenofovir prodrug; and
emtricitabine.

In some aspects, the HIV-infected human, whether diagnosed with a neuropsychiatric and/or neurocognitive complication of HIV infection or predisposed to a neuropsychiatric and/or neurocognitive complication of an HIV infection, is administered:
a darunavir solvate;
cobicistat;
tenofovir disoproxil or a pharmaceutically acceptable salt thereof; and
emtricitabine.

In some aspects, the HIV-infected human, whether diagnosed with a neuropsychiatric and/or neurocognitive complication of HIV infection or predisposed to a neuropsychiatric and/or neurocognitive complication of an HIV infection, is administered:
darunavir ethanolate;
cobicistat;
tenofovir disoproxil fumarate; and
emtricitabine.

In some aspects, the HIV-infected human, whether diagnosed with a neuropsychiatric and/or neurocognitive complication of HIV infection or predisposed to a neuropsychiatric and/or neurocognitive complication of an HIV infection, is administered:
a darunavir solvate;
cobicistat;
tenofovir alafenamide or a pharmaceutically acceptable salt thereof; and
emtricitabine.

In some aspects, the HIV-infected human, whether diagnosed with a neuropsychiatric and/or neurocognitive complication of HIV infection or predisposed to a neuropsychiatric and/or neurocognitive complication of an HIV infection, is administered:
darunavir ethanolate;
cobicistat;
tenofovir alafenamide fumarate; and
emtricitabine.

Darunavir ([(1S,2R)-3-[[(4-aminophenyl)sulfonyl](2-methylpropyl)amino]-2-hydroxy-1-(phenylmethyl)propyl]-carbamic acid (3R,3aS,6aR)-hexahydrofuro[2,3-b] furan-3-yl ester) is a human immunodeficiency virus protease inhibitor and has the following structure:

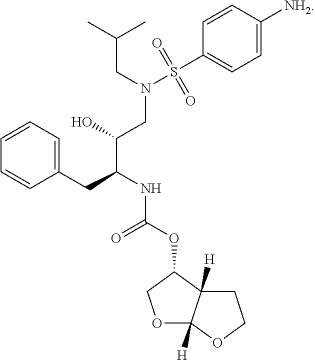 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

According to the disclosure, the human in need of treatment may be administered darunavir.

In other aspects, the human in need of treatment may be administered a pharmaceutically acceptable salt of darunavir.

In other aspects, the human in need of treatment may be administered a darunavir solvate, for example darunavir ethanolate (e.g., PREZISTA®), darunavir propylene glycolate, darunavir glycolate, darunavir propanolate, and the like. For example, in some aspects, the human in need of treatment is administered darunavir ethanolate. In other aspects, the human in need of treatment is administered darunavir propylene glycolate. In other aspects, the human in need of treatment is administered darunavir glycolate. In other aspects, the human in need of treatment is administered darunavir propanolate.

In some aspects, the human in need of treatment may be administered a darunavir hydrate.

According to the disclosure, the human in need is administered the darunavir, the darunavir solvate, or the darunavir hydrate in a therapeutically effective amount. Preferably, the human is administered the therapeutically effective amount, once daily. In other aspects, the human is administered the daily, therapeutically effective amount in divided doses, for example, in two doses per day or in three doses per day. The therapeutically effective amount can be determined by those skilled in the art, and will generally be in the amount equivalent to about 600 mg to about 1000 mg of darunavir, per day. In some aspects, the therapeutically effective amount of the darunavir, the darunavir solvate, or the darunavir hydrate will be an amount equivalent to about 600, 650, 700, 750, 800, 850, 900, 950, or 1000 mg of darunavir, per day. In some aspects, the human in need is administered about 800 mg of darunavir, once daily. For example, the therapeutically effective amount of the darunavir, the darunavir solvate, or the darunavir hydrate will be an amount equivalent to about 600, 650, 700, 750, 800, 850, 900, 950, or 1000 mg of darunavir, once daily.

In some aspects, the human in need is administered about 800 mg of darunavir, per day. In some aspects, the human in need is administered about 800 mg of darunavir, once daily.

In some aspects, the human in need is administered a pharmaceutically acceptable salt of darunavir in an amount equivalent to about 800 mg of darunavir, per day. In some aspects, the human in need is administered a pharmaceutically acceptable salt of darunavir in an amount equivalent to about 800 mg of darunavir, once daily.

In other aspects, the human in need is administered a darunavir solvate, in an amount equivalent to about 800 mg of darunavir, per day. In other aspects, the human in need is administered darunavir ethanolate, in an amount equivalent to about 800 mg of darunavir, per day. In other aspects, the human in need is administered darunavir ethanolate, in an amount equivalent to about 800 mg of darunavir, once daily. In other aspects, the human in need is administered darunavir propylene glycolate, in an amount equivalent to about 800 mg of darunavir, per day. In other aspects, the human in need is administered darunavir propylene glycolate, in an amount equivalent to about 800 mg of darunavir, once daily. In other aspects, the human in need is administered darunavir glycolate, in an amount equivalent to about 800 mg of darunavir, per day. In other aspects, the human in need is administered darunavir glycolate, in an amount equivalent to about 800 mg of darunavir, once daily. In other aspects, the human in need is administered darunavir propanolate, in an amount equivalent to about 800 mg of darunavir, per day. In other aspects, the human in need is administered darunavir propanolate, in an amount equivalent to about 800 mg of darunavir, once daily.

In other aspects, the human in need is administered a darunavir hydrate, in an amount equivalent to about 800 mg of darunavir, per day. In other aspects, the human in need is administered a darunavir hydrate, in an amount equivalent to about 800 mg of darunavir, once daily.

According to the disclosure, the human in need is also administered cobicistat (1,3-thiazol-5-ylmethyl [(2R,5R)-5-{[(2S)-2-[(methyl{[2-(propan-2-yl)-1,3-thiazol-4-yl]methyl}carbamoyl)amino]-4-(morpholin-4-yl)butanoyl]amino}-1,6-diphenylhexan-2-yl]carbamate), a CYP3A inhibitor. Cobicistat has the following structure:

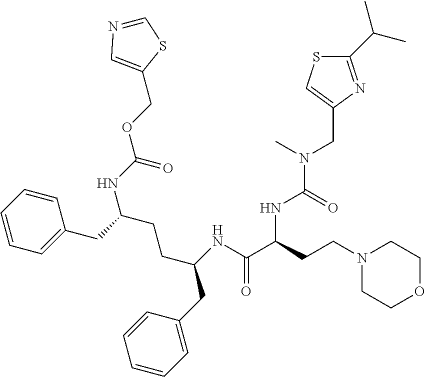 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

According to the disclosure, the human is administered the cobicistat in a therapeutically effective amount. Preferably, the human is administered the therapeutically effective amount, once daily. In other aspects, the human is administered the daily, therapeutically effective amount in divided doses, for example, in two doses per day or in three doses per day. The therapeutically effective amount can be determined by those skilled in the art, and will generally be in the amount equivalent to about 100 mg to about 200 mg of cobicistat, per day. For example, the therapeutically effective amount of cobicistat administered will be an amount equivalent to about 100, 125, 150, 175, or 200 mg of cobicistat, per day. For example, the therapeutically effective amount of cobicistat administered will be an amount equivalent to about 100, 125, 150, 175, or 200 mg of cobicistat, once daily. In some aspects, the human in need is administered about 150 mg of cobicistat, per day. In some aspects, the human in need is administered about 150 mg of cobicistat, once daily.

According to the disclosure, the human in need is also administered tenofovir, a tenofovir prodrug, a pharmaceutically acceptable salt of tenofovir, or a pharmaceutically acceptable salt of a tenofovir prodrug in a therapeutically effective amount. Tenofovir is a nucleotide analog reverse transcriptase inhibitor and has the following structure:

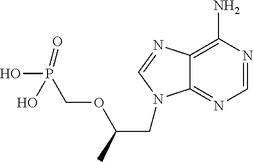 [SMILES: C[C@H](Cn1cnc2c(N)ncnc21)OCP(=O)(O)O]

Preferably, the human is administered the therapeutically effective amount, once daily. In other aspects, the human is administered the daily, therapeutically effective amount in divided doses, for example, in two doses per day or in three doses per day. The therapeutically effective amount of the tenofovir (or a salt thereof) and the tenofovir prodrug (or a salt thereof) can be determined by those skilled in the art.

In some aspects, the human in need is administered tenofovir. According to the disclosure, the human is administered the tenofovir in a therapeutically effective amount. Preferably, the human is administered the therapeutically effective amount, once daily. In other aspects, the human is administered the daily, therapeutically effective amount in divided doses, for example, in two doses per day or in three doses per day. The therapeutically effective amount can be determined by those skilled in the art, and will generally be about 5 mg to about 300 mg of tenofovir, per day. In some aspects, the human is administered about 5 mg to about 300 mg of tenofovir, once daily. For example, the human in need can be administered about 5, 10, 15, 20, 25, 30, 35, 40, 45, 50, 55, 60, 65, 70, 75, 80, 85, 90, 95, 100, 125, 150, 175, 200, 225, 250, 275, or about 300 mg of tenofovir, per day. In some aspects, the human in need is administered about 5, 10, 15, 20, 25, 30, 35, 40, 45, 50, 55, 60, 65, 70, 75, 80, 85, 90, 95, 100, 125, 150, 175, 200, 225, 250, 275, or about 300 mg of tenofovir, once daily.

In some aspects, the human in need is administered a pharmaceutically acceptable salt of tenofovir. According to the disclosure, the human is administered the pharmaceutically acceptable salt of tenofovir in a therapeutically effective amount. Preferably, the human is administered the therapeutically effective amount, once daily. In other aspects, the human is administered the daily, therapeutically effective amount in divided doses, for example, in two doses per day or in three doses per day. The therapeutically effective amount can be determined by those skilled in the art, and will generally be an amount equivalent to about 5 mg to about 300 mg of tenofovir, per day. In some aspects, the human is administered the pharmaceutically acceptable salt of tenofovir in an amount equivalent to about 5 mg to about 300 mg of tenofovir, once daily. For example, the human in need can be administered the pharmaceutically acceptable salt of tenofovir in an amount equivalent to about 5, 10, 15, 20, 25, 30, 35, 40, 45, 50, 55, 60, 65, 70, 75, 80, 85, 90, 95, 100, 125, 150, 175, 200, 225, 250, 275, or about 300 mg of tenofovir, per day. In some aspects, the human in need is administered the pharmaceutically acceptable salt of tenofovir in an amount equivalent to about 5, 10, 15, 20, 25, 30, 35, 40, 45, 50, 55, 60, 65, 70, 75, 80, 85, 90, 95, 100, 125, 150, 175, 200, 225, 250, 275, or about 300 mg of tenofovir, once daily.

In some aspects, the human in need is administered a tenofovir prodrug. Tenofovir prodrugs include, for example, tenofovir alafenamide and tenofovir disoproxil:

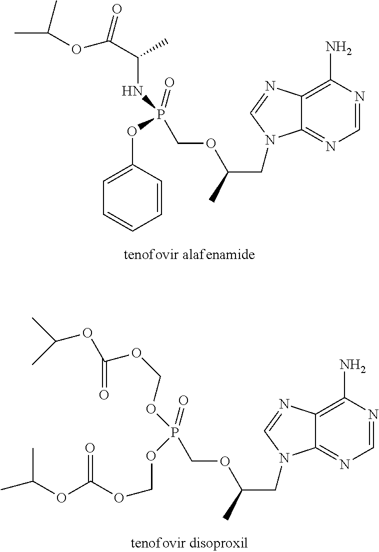 [SMILES: CC(C)OC(=O)[C@H](C)N[P@@](=O)(CO[C@H](C)Cn1cnc2c(N)ncnc21)Oc1ccccc1 ; CC(C)OC(=O)OCOP(=O)(CO[C@H](C)Cn1cnc2c(N)ncnc21)OCOC(=O)OC(C)C]

According to the disclosure, the human in need is administered the tenofovir prodrug in a therapeutically effective amount. Preferably, the human is administered the therapeutically effective amount, once daily. In other aspects, the human is administered the daily, therapeutically effective amount in divided doses, for example, in two doses per day or in three doses per day. The therapeutically effective amount of the tenofovir prodrug can be determined by those skilled in the art, and will generally be in the amount equivalent to about 5 mg to about 300 mg of tenofovir, per day. In other aspects, the therapeutically effective amount of the tenofovir prodrug administered can be determined by those skilled in the art, and will generally be in the amount equivalent to about 5 mg to about 300 mg of the tenofovir prodrug, per day. For example, the therapeutically effective amount of tenofovir prodrug administered will be an amount equivalent to about 5, 10, 15, 20, 25, 30, 35, 40, 45, 50, 55, 60, 65, 70, 75, 80, 85, 90, 95, 100, 125, 150, 175, 200, 225, 250, 275, or 300 mg of tenofovir, per day. In some aspects, the therapeutically effective amount of tenofovir prodrug administered will be an amount equivalent to about 5, 10, 15, 20, 25, 30, 35, 40, 45, 50, 55, 60, 65, 70, 75, 80, 85, 90, 95, 100, 125, 150, 175, 200, 225, 250, 275, or 300 mg of tenofovir, once daily. In some aspects, the therapeutically effective amount of tenofovir prodrug administered will be an amount equivalent to about 5, 10, 15, 20, 25, 30, 35, 40, 45, 50, 55, 60, 65, 70, 75, 80, 85, 90, 95, 100, 125, 150, 175, 200, 225, 250, 275, or 300 mg of tenofovir prodrug, per day. In some aspects, the therapeutically effective amount of tenofovir prodrug administered will be an amount equivalent to about 5, 10, 15, 20, 25, 30, 35, 40, 45, 50, 55, 60, 65, 70, 75, 80, 85, 90, 95, 100, 125, 150, 175, 200, 225, 250, 275, or 300 mg of tenofovir prodrug, once daily.

In some aspects, the human in need may be administered a pharmaceutically acceptable salt a tenofovir prodrug. Preferably, the human is administered a therapeutically effective amount of the pharmaceutically acceptable salt of the tenofovir prodrug, once daily. In other aspects, the human is administered the daily, therapeutically effective amount of the pharmaceutically acceptable salt of the tenofovir prodrug in divided doses, for example, in two doses per day or in three doses per day.

For example, the human in need may be administered a pharmaceutically acceptable salt of tenofovir alafenamide, for example tenofovir alafenamide fumarate. When administering tenofovir alafenamide fumarate, a therapeutically effective amount will be an amount equivalent to about 10 mg to about 25 mg of tenofovir alafenamide, per day. In some aspects, the tenofovir alafenamide fumarate will be administered in an amount equivalent to 10, 11, 12, 13, 14, 15, 16, 17, 18, 19, 20, 21, 22, 23, 24, or about 25 mg of tenofovir alafenamide, per day. For example, the tenofovir alafenamide fumarate will be administered in an amount equivalent to 10, 11, 12, 13, 14, 15, 16, 17, 18, 19, 20, 21, 22, 23, 24, or about 25 mg of tenofovir alafenamide, once daily.

In other aspects, the human in need may be administered a pharmaceutically acceptable salt of tenofovir disoproxil, for example tenofovir disoproxil fumarate. When administering tenofovir disoproxil fumarate, a therapeutically effective amount will be about 150 mg to about 300 mg of tenofovir disoproxil fumarate, per day. For example, the tenofovir disoproxil fumarate will be administered in an amount equivalent to about 150, 160, 170, 180, 190, 200, 210, 220, 230, 240, 250, 260, 270, 280, 290, or about 300 mg of tenofovir disoproxil fumarate, per day. In some aspects, the tenofovir disoproxil fumarate will be administered in an amount equivalent to about 150, 160, 170, 180, 190, 200, 210, 220, 230, 240, 250, 260, 270, 280, 290, or about 300 mg of tenofovir disoproxil fumarate, once daily.

According to the disclosure, the human in need is also administered emtricitabine in a therapeutically effective amount. Preferably, the human is administered the therapeutically effective amount, once daily. In other aspects, the human is administered the daily, therapeutically effective amount in divided doses, for example, in two doses per day or in three doses per day. The therapeutically effective amount can be determined by those skilled in the art, and will generally be about 150 mg to about 200 mg of emtricitabine, per day. For example, the therapeutically effective amount of the emtricitabine will be about 150, 160, 170, 180, 190, or about 200 mg of emtricitabine, per day. In some aspects, the therapeutically effective amount of the emtricitabine will be about 150, 160, 170, 180, 190, or about 200 mg of emtricitabine, once daily.

According to the disclosure, each of the described active agents (i.e., darunavir, a darunavir solvate, or a darunavir hydrate; cobicistat; tenofovir, a tenofovir prodrug, a pharmaceutically acceptable salt of tenofovir, or a pharmaceutically acceptable salt of a tenofovir prodrug; and emtricitabine) can be administered as individual, single unit dosage forms. In these aspects, the human in need is administered
a single unit dosage form of darunavir, a darunavir solvate, or a darunavir hydrate;
a single unit dosage form of cobicistat;
a single unit dosage form of tenofovir, a tenofovir prodrug, a pharmaceutically acceptable salt of tenofovir, or a pharmaceutically acceptable salt of a tenofovir prodrug; and
a single unit dosage form of emtricitabine.

The single unit dosage forms are preferably each administered once daily. In other aspects, each active agent can be administered to the human in the form of a single unit dosage form, two or more times per day.

For example, darunavir ethanolate is available in single unit dosage forms under the tradename PREZISTA® (darunavir ethanolate equivalent to 800 mg of darunavir), among others. Cobicistat (absorbed onto silicon dioxide) is available in single unit dosage forms under the tradename TYBOST® (150 mg cobicistat), among others. Tenofovir disoproxil fumarate is available in single unit dosage forms under the tradename VIREAD® (150, 200, 250, and 300 mg of tenofovir disoproxil fumarate), among others. Tenofovir alafenamide fumarate is available in single unit dosage forms under the tradename VEMLIDY® (tenofovir alafenamide fumarate equivalent to 25 mg of tenofovir alafenamide), among others. Emtricitabine is available in single unit dosage forms under the tradename EMTRIVA® (200 mg emtricitabine), among others.

Alternatively, each of the active agents can be combined with one or more of the other active agents in a single unit dosage form containing two or more of the described active agents.

For example, the human in need may be administered:

a single unit dosage form comprising darunavir, a darunavir solvate, or a darunavir hydrate; and cobicistat; and

a single unit dosage form comprising tenofovir, a tenofovir prodrug, a pharmaceutically acceptable salt of tenofovir, or a pharmaceutically acceptable salt of a tenofovir prodrug; and

a single unit dosage form emtricitabine.

In other aspects, the human in need may be administered:

a single unit dosage form comprising darunavir, a darunavir solvate, or a darunavir hydrate;

a single unit dosage form comprising cobicistat; and

a single unit dosage form comprising tenofovir, a tenofovir prodrug, a pharmaceutically acceptable salt of tenofovir, or a pharmaceutically acceptable salt of a tenofovir prodrug; and emtricitabine.

In other aspects, the human in need may be administered:

a single unit dosage form comprising darunavir, a darunavir solvate, or a darunavir hydrate; and cobicistat; and

a single unit dosage form comprising tenofovir, a tenofovir prodrug, a pharmaceutically acceptable salt of tenofovir, or a pharmaceutically acceptable salt of a tenofovir prodrug; and emtricitabine.

In other aspects, the human in need may be administered:

a single unit dosage form comprising darunavir, a darunavir solvate, or a darunavir hydrate; cobicistat; tenofovir, a tenofovir prodrug, a pharmaceutically acceptable salt of tenofovir, or a pharmaceutically acceptable salt of a tenofovir prodrug; and emtricitabine.

For example, the darunavir, the darunavir solvate, or the darunavir hydrate can be combined with cobicistat in a single unit dosage form comprising cobicistat and darunavir, a darunavir solvate, or a darunavir hydrate. In some aspects, cobicistat is combined with darunavir in a single unit dosage form comprising cobicistat and darunavir. In other aspects, cobicistat is combined with a darunavir solvate in a single unit dosage form comprising cobicistat and a darunavir solvate, for example, darunavir ethanolate. Cobicistat and darunavir ethanolate is available in single unit dosage forms under the tradenames PREZCOBIX® (darunavir ethanolate equivalent to 800 mg of darunavir and 150 mg cobicistat) and REZOLSTA® (darunavir ethanolate equivalent to 800 mg of darunavir and 150 mg cobicistat), among others.

In yet other aspects cobicistat is combined with a darunavir hydrate in a single unit dosage form comprising cobicistat and a darunavir hydrate.

In other exemplary embodiments, the emtricitabine and tenofovir, a tenofovir prodrug, a pharmaceutically acceptable salt of tenofovir, or a pharmaceutically acceptable salt of a tenofovir prodrug can be combined in a single unit dosage form comprising emtricitabine and tenofovir, a tenofovir prodrug, a pharmaceutically acceptable salt of tenofovir, or a pharmaceutically acceptable salt of a tenofovir prodrug. In some aspects, emtricitabine is combined with tenofovir in a single unit dosage form comprising emtricitabine and tenofovir. In other aspects, emtricitabine is combined with a pharmaceutically acceptable salt of tenofovir in a single unit dosage form comprising emtricitabine and a pharmaceutically acceptable salt of tenofovir. In other aspects, emtricitabine is combined with a tenofovir prodrug in a single unit dosage form comprising emtricitabine and a tenofovir prodrug. In other aspects, emtricitabine is combined with a pharmaceutically acceptable salt of a tenofovir prodrug in a single unit dosage form comprising emtricitabine and a pharmaceutically acceptable salt of a tenofovir prodrug. Emtricitabine and tenofovir disoproxil fumarate is available in single unit dosage forms under the tradename TRUVADA® (200 mg of emtricitabine and 300 mg of tenofovir disoproxil fumarate), among others. Emtricitabine and tenofovir alafenamide fumarate is available in single unit dosage forms under the tradename DESCOVY® (200 mg of emtricitabine and 28 mg tenofovir alafenamide fumarate (equivalent to 25 mg of tenofovir alafenamide)), among others.

In other exemplary embodiments, the darunavir, a darunavir solvate, or a darunavir hydrate; cobicistat; tenofovir, a tenofovir prodrug, a pharmaceutically acceptable salt of tenofovir, or a pharmaceutically acceptable salt of a tenofovir prodrug; and emtricitabine can be combined together into a single unit dosage form. For example, a single unit dosage form of the disclosure may comprise a darunavir solvate, a pharmaceutically acceptable salt of a tenofovir prodrug, cobicistat, and emtricitabine. An exemplary single unit dosage form is available under the tradename SYMTUZA®, (darunavir ethanolate (equivalent to 800 mg of darunavir), cobicistat (150 mg) absorbed onto silicon dioxide, tenofovir alafenamide fumarate (equivalent to 10 mg of tenofovir alafenamide), and emtricitabine (200 mg)) and SYMTUZA® (darunavir ethanolate (equivalent to 800 mg of darunavir), cobicistat (150 mg) absorbed onto silicon dioxide, tenofovir alafenamide fumarate (equivalent to 10 mg of tenofovir alafenamide), and emtricitabine (200 mg)), among others.

In some aspects, the single unit dosage forms of the disclosure may be used in combination with additional active ingredients in the treatment of HIV infections. The additional active ingredients may be co-administered separately with a single unit dosage form of the disclosure. The combination may serve to increase efficacy (e.g., by including in the combination a compound potentiating the potency or effectiveness of an active agent according to the disclosure), decrease one or more side effects, or decrease the required dose of the active agent according to the disclosure.

Oral dosage forms, for example, tablets, of the disclosure may include an active agent according to the disclosure mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, microcrystalline cellulose, silicified microcrystalline cellulose, mannitol, sorbitol, and the like. Starch, polyvinyl-pyrrolidone (PVP), sodium starch glycolate, microcrystalline cellulose, carboxymethylcellulose, and alginic acid are suitable disintegrating agents. Binding agents may include starch and gelatin. The lubricating agent, if present, may be magnesium stearate, stearic acid, sodium stearyl fumarate, or talc. If desired, the tablets may be coated with an esthetic coating based on, for example, hydroxypropyl methyl cellulose or polyvinyl alcohol copolymers together with wetting, anti-tacking, and/or coloring agents, and the like. If desired, the tablets may be coated with materials such as, for example, glyceryl monostearate or glyceryl distearate to delay absorption in the gastrointestinal tract, or may be coated with an enteric coating.

In addition to tablets, the single unit dosage forms of the disclosure may be presented as capsules, for example, hard and soft gelatin capsules. To prepare hard gelatin capsules, active agents of the disclosure of the disclosure may be mixed with a solid, semi-solid, or liquid diluent. Soft gelatin capsules may be prepared by mixing an active agent of the disclosure with water, an oil such as peanut oil or olive oil, liquid paraffin, a mixture of mono and di-glycerides of short chain fatty acids, polyethylene glycol 400, or propylene glycol.

Manufacturing a multi-component, single unit dosage form, in particular one that comprises four active agents, wherein at least one of those agents must be present in large amounts, for example, about 400 to about 1200 mg, preferably about 800 mg in the single unit dosage form, is challenging. One particular challenge is manufacturing a multi-component formulation that accommodates large dosage ranges and keeps the dosage form small enough for a subject to swallow. Single unit dosage forms of the disclosure can be prepared according to methods known in the art, for example, methods described in WO2013/004816 and WO2013004818, the entireties of which are incorporated by reference herein.

In preferred embodiments, the single unit dosage forms of the disclosure, when in the form of tablets, are of a sufficient structural integrity that they can be split, preferably into two or more pieces, for those subjects who have difficulties in swallowing larger-sized tablets. Score lines may be implemented to aid in splitting of the dosage forms.

According to the methods disclosed herein, an improvement in one or more of the neuropsychiatric and/or neurocognitive complications of an HIV infection will occur with the administration of the described active agents to the human diagnosed with an HIV infection, for example, an HIV-1 infection, who is also diagnosed with one or more neuropsychiatric and/or neurocognitive complications of an HIV infection.

Improvements in neuropsychiatric and neurocognitive complications of an HIV infection can be measured using methods known in the art. For example, improvements in neurocognitive complications of an HIV infection can be measured using the Montreal Cognitive Assessment (MoCA), whereas improvements in neuropsychiatric complications can be measured using the Hospital Anxiety and Depression Scale (HADS), the Pittsburgh Sleep Quality Index (PSQI), the Fatigue Severity Scale (FSS), or a combination of the preceeding. In some aspects, the improvement is measured using MoCA. In some aspects, the improvement is measured using HADS. In some aspects, the improvement is measured using PSQI. In some aspects, the improvement is measured using FSS. These improvements could be adjusted using the 10 most prevalent stressful life events an HIV patient may experience from the modified SLE-Q, according to Corless et al.

The following examples are merely illustrative and are not intended to limit the disclosure to the materials, conditions, or process parameters set forth therein.

### Examples

### Objectives

The study described herein has several objectives, including, but not limited to, the evaluation of the differences in cognitive function, depression, anxiety, fatigue, and quality of sleep from baseline to 12 weeks and/or time of discontinuation in HIV positive, ART-naïve patients receiving DRV/COBI/FTC/TAF (darunavir/cobicistat/emtricitabine/tenofovir alafenamide fumarate) or DRV/COBI (darunavir/cobicistat) plus TDF/FTC (tenofovir diisoproxil fumarate/emtricitabine) or TAF/FTC (tenofovir alafenamide fumarate/emtricitabine) as first-line treatment.

Study objectives also include evaluating:
Overtime differences in cognitive function, depression, anxiety, fatigue, and quality of sleep from baseline to 12, 24, 48 weeks and/or time of discontinuation.
Factors potentially associated with changes in neurocognitive functions, quality of sleep, fatigue, depression, and anxiety.
Prevalence of neurocognitive impairment, quality of sleep, fatigue, depression, anxiety in naïve HIV patients.
Prevalence of first-line treatment discontinuation and reasons for discontinuation.
Proportion of patients with virological suppression (HIV-1 RNA levels <50 copies/mL) and differences in CD4+ cell counts from baseline to 12, 24 and 48 weeks and/or time of discontinuation.
Comparison of cognitive function, depression, anxiety, fatigue and quality of sleep indices between those patients who receive depression treatment during the course of the study and those who do not.

### Study Design and Rationale

This is an observational, prospective, multicenter, single-arm, non-interventional study evaluating the differences in neurocognitive function and mental health outcomes (depression, anxiety, fatigue and quality of sleep) in 200 HIV positive, ART-naïve patients receiving DRV/cobi/FTC/TAF or DRV/cobi plus TDF/FTC or TAF/FTC according to clinical practice, during their first year of treatment. Approximately 16 sites will participate across Greece.

Only data that will become available per clinical practice will be collected within this study. No laboratory, diagnostic or therapeutic procedures other than those currently performed as part of routine care will be required per protocol. Additionally, as permitted in accordance with local regulations, participating physicians will be asked to obtain patient-reported outcome (PRO) data from patients within this study.

The decision to treat with DRV/COBI/FTC/TAF or DRV/cobi plus TDF/FTC or TAF/FTC must have been freely undertaken by the clinician prior to the decision of patients to participate in this observational study. Prior to data collection, all patients must sign a participation agreement/informed consent form (ICF) allowing data collection and source data verification in accordance with local requirements and/or sponsor policy. For practical reasons and to avoid the burden of additional visits and avoid interfering with routine clinical practice, it is allowed for the ICF date to be the same as the date of first visit and decision to treat the patient with DRV/cobi/FTC/TAF or DRV/cobi plus TDF/FTC or TAF/FTC, and within 30 days after the first dose.

A patient's baseline data will be collected, where available, at initiation of treatment with DRV/cobi/FTC/TAF or DRV/cobi plus TDF/FTC or TAF/FTC, and no more than 30 days after the first dose of DRV/cobi/FTC/TAF or DRV/cobi plus TDF/FTC or TAF/FTC. This will be followed by an observational period during which data will be collected in 12-weekly intervals (±2 weeks) until the final visit at 48 weeks (±2 weeks), or time of discontinuation/switch of ART. The end of the study will be the last data collection point within the study for the last participating patient.

Data collection will include details of the patient's demographics, disease characteristics, possible mode of HIV transmission, educational level, employment status, place of residence, type of residence (urban, suburban, rural), past and concurrent medical diseases and medication, smoking, alcohol and/or illicit drug use. Patient's baseline viral load and CD4+ T-cell count will be recorded, per routine clinical practice. Additionally, neurocognitive function will be evaluated by the physician's completion of the Montreal Cognitive Assessment (MoCA) questionnaire. Three self-completed neuropsychiatric scales will also be completed by participants of the study: Hospital Anxiety and Depression Scale (HADS) to evaluate patient's anxiety and depression; Pittsburgh Sleep Quality Index (PSQI) to evaluate quality of sleep; and Fatigue Severity Scale (FSS) to evaluate fatigue. Finally, only the 10 most prevalent possible on going stressful and traumatic life events frequently experienced among HIV-infected individuals will be recorded, according to Corless et al. Additionally, where applicable, reasons for discontinuation of current regimen and details of the new regimen will be recorded.

### Study Setting and Patient Population

Patients participating in this non-interventional study will be treated with DRV/cobi/FTC/TAF or DRV/cobi plus TDF/FTC or TAF/FTC as treatment for HIV in accordance with clinical practice. Approximately 16 sites will participate across Greece, providing a representative sample of the HIV population in Greece. The final numbers and locations of sites will be decided upon feasibility assessments.

Participating sites will be encouraged to enroll patients in a consecutive manner when patients come for their regular visit, in order to minimize bias in patient selection. At each participating site, the participating physician will assess patients to determine their eligibility for data collection within the study based on the following selection criteria: males and females aged 18 years of age, inclusive who have confirmed diagnosis of HIV, are ART-naïve and eligible to receive DRV/cobi/FTC/TAF or DRV/cobi plus TDF/FTC or TAF/FTC per clinical decision.

Prior to data collection, all patients must sign a participation agreement/ICF allowing data collection and source data verification in accordance with local requirements and/or sponsor policy. The time of ICF signature must be after the clinical decision to treat the patient with DRV/cobi/FTC/TAF or DRV/cobi plus TDF/FTC or TAF/FTC and within 30 days after the first dose.

### Data Sources and Collection Methods

The primary data source for this study will be the medical records of each patient. Source documentation should be in patients' medical records for all data entered into the Case Report Form (CRF). Additionally, patient- and physician-completed questionnaires, scales, and assessments will be recorded onto paper forms and will be considered source data.

### Measures of Effectiveness/Clinical Response and Patient Reported Outcomes

Data from laboratory reports done on blood samples collected during routine clinical practice evaluating the HIV-1 RNA levels and CD4+ T-cell count will be collected. Neurocognitive function will be evaluated by the physician's completion of the MoCA questionnaire. Where permitted, participating physicians will be asked to obtain PRO data from patients participating in this observational study. The following measures will be recorded:
HADS will be used to measure anxiety and depression.
PSQI will be used to evaluate quality of sleep.
FSS will be used to assess and quantify fatigue.
From the modified SLE-Q, only the 10 most frequent stressful life events will be captured, as identified by Corless et al, 2013.

### Patient Reported Outcomes

HIV is associated with psychological comorbidities that have a significant impact on the patient's life and include depression, anxiety, sleep disturbances, and fatigue. Data on PROs related to these comorbidities will be collected regarding associations between HIV and these comorbidities.

Anxiety and depression will be assessed using the HADS questionnaire. The HADS is a tool to measure anxiety and depression that has been translated and validated in many countries since it's development in 1983, and that has been shown to be reliable in detection of anxiety and depression in patients.

Quality of sleep in patients will be evaluated using the PSQI. The PSQI questionnaire is widely used clinically as a simple and valid assessment of both sleep quality and disturbance that might affect sleep quality.

The FSS will be completed by patients to measure fatigue in this study. The FSS is a validated 9-item self-report questionnaire that is frequently used to assess and quantify fatigue.

From the modified SLE-Q will be recorded only the 10 most prevalent possible on going stressful and traumatic life events frequently experienced among HIV-infected individuals. This instrument is modified from the 12-item List of Threatening Experiences questionnaire, which was originally validated with psychiatric patients and shown to have a high test-retest reliability. Only the 10 most frequent stressful life events that may have been experienced by patients within the previous month will be recorded, as identified by Corless et al, 2013.

All the rating scales that refer to the study endpoints have previously been used across HIV positive populations in numerous clinical studies, and have been translated in Greek and validated for the Greek population (albeit for different diagnoses than HIV infection).

### Patient Selection Criteria

Participating sites will be encouraged to enroll patients in a consecutive manner when patients come for their regular visit, in order to minimize bias in patient selection. All eligible patients should be offered enrollment for data collection within the study.

At each participating site, the participating physician will assess patients to determine their eligibility for data collection within the study based on the selection criteria listed below. If there is a question about any of the selection criteria, the participating physician should consult with the appropriate sponsor representative before enrolling the patient.

Each potential patient must satisfy the following criteria to be eligible for data collection in this study:

1. male or female aged 18 years of age, inclusive.

2. must have a confirmed diagnosis of HIV.

3. must be ART-naïve.

4. must be eligible to receive DRV/cobi/FTC/TAF or DRV/cobi plus TDF/FTC or TAF/FTC per clinical decision.

5. must sign a participation agreement/ICF allowing data collection and source data verification in accordance with local requirements

6. the time of participation agreement/ICF signature must be after the clinical decision to treat the patient with DRV/cobi/FTC/TAF or DRV/cobi plus TDF/FTC or TAF/FTC, and within 30 days after the first dose.

Potential patients who meet any of the following criteria will not be eligible for this study:

1. is a woman who is pregnant, or breast-feeding, or planning to become pregnant while enrolled in this study

2. current intravenous drug use and/or intravenous drug use within the past 6 months prior to study enrollment.

3. diagnosis of any major psychiatric disorder (ie, bipolar disorder, psychosis, etc) and/or administration of antidepressant, anxiolytics, hypnotics and other psychiatric medication at the time of enrollment.

4. participation in any interventional clinical study (currently or during the 6 months prior to enrollment in the study).

5. compromised ability to give informed consent (defined per clinical judgement).

### Effectiveness/Clinical Response Measures

Viral Load and Immunology

Results from laboratory testing done under routine clinical practice (HIV-1 RNA levels and CD4+ T-cell count) will be recorded in the study CRF at the visits indicated in the DATA COLLECTION SCHEDULE, as available in the patient medical record. No repeat or additional testing will be done for the purposes of this study. Virological suppression will be defined as HIV RNA <50 copies/mL.

Montreal Cognitive Assessment

Neurocognitive function will be evaluated by the physician's completion of the MoCA questionnaire at the visits. The MoCA takes approximately 10 minutes to complete and consists of 30 items that encompass 8 cognitive domains. The domains of impairment tested include executive functioning (assessed by alternating trail making test, phonemic fluency and a 2-item abstraction task); visuospatial ability (evaluated by a clock-drawing task and 3-dimensional cube copy); language (assessed by sentence repetition, phonemic fluency and a naming task); orientation (assessed through correct identification of the current date, location, and city; short term memory (assessed through a 5 minute delayed recall); 4 subtests are used to assess attention/working memory: digit spans forward and backward, tapping test and a serial 7 subtraction task.

### Patient Reported Outcomes

Patient-reported outcomes that are available within clinical practice will be collected. Additionally, participating physicians will be asked to obtain PROs from patients within this study. Questionnaires should be completed by the patient at each applicable occasion. Where possible, PROs should be completed before other assessments. The following measures will be recorded:

Hospital Anxiety and Depression Scale (Anxiety [-A], Depression [-D])

The HADS is a validated 14-item scale with 7 of the items relating to anxiety and 7 relating to depression. Each item is scored from 0 to 3, with higher scores indicating greater likelihood of depression or anxiety. Cases of anxiety or depression are each defined by subscale scores of 8 or greater. The recall period is the past week. The HADS takes approximately 2 to 5 minutes to complete.

Pittsburgh Sleep Quality Index

The PSQI is a validated 19-item questionnaire to produce a global sleep quality score. The instrument consists of 7 component scores including sleep quality, sleep latency, sleep duration, habitual sleep efficiency, sleep disturbance, use of sleeping medications, and daytime dysfunction. These 7 components produce a global score ranging from 0 to 21 with a lower score indicating healthier sleep quality and a score of 5 or above indicative of poor sleep quality.

Fatigue Severity Scale

The FSS is a validated 9-item self-report questionnaire that is frequently used to assess and quantify fatigue. Each item in the FSS ranges from 1 to 7, where 1 indicates strong disagreement and 7 indicates strong agreement. The final score represents the mean value of the 9 items.

Stressful Life Events Questionnaire

The SLE-Q is a validated 20-item instrument, from which only the 10 most frequent stressful life events that a HIV patient may have experienced in the previous month, as identified by Corless et al, 2013, will be captured. The items include financial problems, unemployment, loss of a close friend or relative, alcohol/drug abuse, no food due to lack of money, rejection associated with HIV diagnosis, hospitalization, breaking off a steady relationship, loss of something valuable and serious problems with a close friend, relative or neighbor. Scoring for each item is binary (yes, no). Answers for these items will be captured by the treating physician after interviewing the patient during the scheduled clinical visits.

### Effectiveness/Clinical Response Analysis

The following objectives will be assessed using descriptive techniques like proportions:
Prevalence of neurocognitive impairment, quality of sleep, fatigue, depression, anxiety in naïve HIV patients.
Prevalence of first-line treatment discontinuation and reasons for discontinuation.
Proportion of patients with virological suppression (HIV-1 RNA levels <50 copies/mL) and differences in CD4+ cell counts from baseline to 12, 24 and 48 weeks and/or time of discontinuation.

The objective to compare cognitive function, depression, anxiety, fatigue and quality of sleep indices between those patients who receive depression treatment during the course of the study and those who do not, will be assessed by considering the cross-sectional effect of the time dependent indicator which describes if a patient receives depression treatment or not at each visit. This indicator will be included in all models described above for assessing the objective. Data will be collected from all patients who meet the eligibility criteria.

1. A method of treating a human diagnosed with an HIV infection and
diagnosed with a neuropsychiatric and/or a neurocognitive complication of HIV infection; or
predisposed to a neuropsychiatric and/or a neurocognitive complication of HIV infection
comprising
administering to the human
darunavir, a darunavir solvate, or a darunavir hydrate;
cobicistat;
tenofovir, a tenofovir prodrug, a pharmaceutically acceptable salt of tenofovir, or a pharmaceutically acceptable salt of a tenofovir prodrug; and
emtricitabine.

2. The method of claim 1, wherein the neuropsychiatric complication of HIV infection is depressive spectrum disorders including major depressive disorder, anxiety including generalized anxiety disorder, social phobia, fatigue including chronic fatigue, sleep disturbance including disorders of initiating and/or maintaining sleep, disorders of excessive somnolence, disorders of sleep-wake schedule, and dysfunctions associated with sleep, sleep stages, or partial arousals, or poor sleep quality, or a combination thereof.

3. The method of claim 1, wherein the neurocognitive complication of HIV infection is asymptomatic neurocognitive impairment, mild neurocognitive disorder, or HIV-associated dementia (HAD).

4. The method of claim 1, comprising administration of a tenofovir prodrug or a pharmaceutically acceptable salt of a tenofovir prodrug.

5. The method of claim 4, wherein the tenofovir prodrug is tenofovir disoproxil.

6. The method of claim 4, wherein the pharmaceutically acceptable salt of a tenofovir prodrug is tenofovir disoproxil fumarate.

7. The method of claim 4, wherein the tenofovir prodrug is tenofovir alafenamide.

8. The method of claim 4, wherein the pharmaceutically acceptable salt of a tenofovir prodrug is tenofovir alafenamide fumarate.

9. The method of claim 4, wherein the human is administered darunavir, a darunavir hydrate, or a darunavir solvate;
cobicistat;
a tenofovir prodrug or a pharmaceutically acceptable salt thereof; and
emtricitabine.

10. The method of claim 4, wherein the human is administered
a darunavir solvate,
cobicistat,
tenofovir disoproxil or a pharmaceutically acceptable salt thereof, and
emtricitabine.

11. The method of claim 10, wherein the human is administered
darunavir ethanolate,
cobicistat,
tenofovir disoproxil fumarate, and
emtricitabine.

12. The method of claim 10, wherein the darunavir solvate and cobicistat are administered as a single unit dosage form comprising the darunavir solvate and cobicistat.

13. The method of claim 10, wherein the tenofovir disoproxil or a pharmaceutically acceptable salt thereof and emtricitabine are administered as a single unit dosage form comprising the tenofovir disoproxil or a pharmaceutically acceptable salt thereof and emtricitabine.

14. The method of claim 1, wherein the human is administered
a darunavir solvate,
cobicistat,
tenofovir alafenamide or a pharmaceutically acceptable salt thereof, and
emtricitabine.

15. The method of claim 14, wherein the human is administered
darunavir ethanolate,
cobicistat,
tenofovir alafenamide fumarate, and
emtricitabine.

16. The method of claim 14, wherein the darunavir solvate and cobicistat are administered as a single unit dosage form comprising the darunavir solvate and cobicistat.

17. The method of claim 14, wherein the tenofovir alafenamide or a pharmaceutically acceptable salt thereof and emtricitabine are administered as a single unit dosage form comprising the tenofovir alafenamide or a pharmaceutically acceptable salt thereof and emtricitabine.

18. The method claim 1, wherein the darunavir, darunavir solvate, or darunavir hydrate; cobicistat; tenofovir, tenofovir prodrug, or pharmaceutically acceptable salt thereof; and emtricitabine are administered as a single unit dosage form.

19. The method of claim 18, wherein the human is administered darunavir ethanolate in an amount equivalent to about 800 mg of darunavir, once daily.

20. The method claim 18, wherein the human is administered about 150 mg of cobicistat, once daily.

21. The method of claim 18, wherein the human is administered about 150 mg to about 200 mg of emtricitabine, once daily.

22. The method claim 18, wherein the human is administered tenofovir alafenamide fumarate in an amount equivalent to about 10 mg to about 25 mg of tenofovir alafenamide, once daily.

23. The method of claim 1, wherein the human is administered about 150 mg to about 300 mg of tenofovir disoproxil fumarate, once daily.

24. The method claim 23, wherein the human is diagnosed with an HIV infection and diagnosed with a neuropsychiatric complication of HIV infection.

25. The method of claim 1, wherein the human is diagnosed with an HIV infection and diagnosed with a neurocognitive complication of HIV infection.

26. The method of claim 1, wherein the human is diagnosed with an HIV infection and is predisposed to a neuropsychiatric complication of HIV infection.

27. The method of claim 26, wherein the human is diagnosed with an HIV infection and is predisposed to a neurocognitive complication of HIV infection.

28. The method of claim 27, wherein the HIV infection is an HIV-1 infection.

29. The method of claim 28, wherein the human is ART-naïve prior to the administration.

30. The method of claim 29, wherein the administration results in an improvement in a neuropsychiatric complication in the human.

31. The method of claim 30, wherein the administration results in an improvement in a neurocognitive complication in the human.

32. The method of claim 30, wherein the improvement is measured using a Montreal Cognitive Assessment (MoCA), a Hospital Anxiety and Depression Scale (HADS), a Pittsburgh Sleep Quality Index (PSQI), a Fatigue Severity Scale (FSS), or a combination thereof.
